(19) United States
(12) Patent Application Publication
(10) Pub. No.: US 2025/0050314 A1
(43) Pub. Date: Feb. 13, 2025

(54) SHELL CATALYST FOR PRODUCING ALKENYL CARBOXYLIC ACID ESTERS HAVING AN IMPROVED PD AND AU DISTRIBUTION

(71) Applicant: Clariant International Ltd, Muttenz (CH)

(72) Inventors: Roman BOBKA, Rosenheim (DE); Gerhard MESTL, München (DE); Peter SCHECK, Gilching Bavaria (DE)

(21) Appl. No.: 18/718,546

(22) PCT Filed: Dec. 9, 2022
(86) PCT No.: PCT/EP2022/085191

§ 371 Date: Jun. 11, 2024

(30) Foreign Application Priority Data
Dec. 21, 2021 (EP) 21216647.4

(51) Int. Cl. B01J 23/52; B01J 31/04; B01J 35/30; B01J 37/02; B01J 37/18; C07C 67/055

(52) U.S. Cl. CPC B01J 23/52; B01J 31/04; B01J 35/394; B01J 35/397; B01J 37/0207; B01J 37/0236; B01J 37/024; B01J 37/18; C07C 67/055; B01J 2531/13

(57) ABSTRACT

The present invention relates to a Pd- and Au-containing shell catalyst which is characterised by an improved Pd and Au distribution. The invention also relates to two methods for producing said catalyst and to a method for producing vinyl acetate monomer using said catalyst.

The present invention relates to a Pd- and Au-containing shell catalyst which is characterized by an improved distribution of Pd and Au. The invention also relates to two processes for producing this catalyst and to a process for producing vinyl acetate monomer using this catalyst.

Shell catalysts have been used for years in various catalytic processes. They have the feature that the catalytically active species are not distributed over the entire catalyst support, but only in a shell-like region around the center of the catalyst support.

One field of application is the production of vinyl acetate monomer (VAM) by reaction of ethylene, acetic acid and oxygen. Shell catalysts which contain Pd and Au compounds as catalytically active species and can also contain various promoters are widely employed here.

Thus, WO 2008/145388 A1 describes the production of Pd- and Au-containing shell catalysts where the application of the catalytically active species is carried out by spray impregnation of a fluidized bed of catalyst supports. The resulting shell-like regions of the catalytically active species have a largely constant distribution of the catalytically active species.

In WO 2005/065821 A1 the pore structure of catalyst supports is loaded by absorption of a solution containing the catalytically active species. Alternatively a mixture of catalytically active species and binder material is applied to a surface of the catalyst support. The distribution of the catalytically active species is disadvantageous because it either extends relatively far into the interior of the support or an outer layer where the distribution is very largely homogeneous over a relatively wide region of this applied shell is produced.

There further remains a need for Pd- and Au-containing shell catalysts where these two catalytically active species are present in a relatively narrow layer and have a concentration profile which is as similar as possible, in particular maxima of the concentration distribution which are as similar as possible, in order thus to achieve an improved catalytic effect.

This object is achieved by a Pd- and Au-containing shell catalyst which is characterized by a concentration profile of the palladium and a concentration profile of the gold within the shell catalyst where the maxima of the palladium and gold concentrations are disposed in a range from 0 to 40 micrometers from the geometric surface of the catalyst shaped body and the distance between the maximum of the palladium concentration and the maximum of the gold concentration is in the range from 0 to 10 micrometers.

The present invention further provides a first process for producing the shell catalyst according to the invention. This comprises the steps of:
(a) subjecting a bed of a catalyst support to a recirculating motion, wherein the bed is heated to a temperature of 60° C. to 120° C. during subjection to the recirculating motion,
(b) sequential or simultaneous application of a dissolved Pd-containing precursor compound and a dissolved Au-containing precursor compound by spray application onto the bed of the catalyst support subjected to the recirculating motion, wherein in the case of simultaneous application the temperature is 5° C. to 30° C., preferably 7° C. to 25° C., most preferably 10° C. to 20° C., below the temperature established in step (a) and wherein in the case of sequential application the temperature of the first application is 5° C. to 30° C., preferably 7° C. to 25° C., most preferably 10° C. to 20° C., below the temperature established in step (a).

The catalyst according to the invention is also producible by a second process according to the invention. This comprises the steps of:
(a) subjecting a bed of a catalyst support to recirculating motion and contacting the bed with atomized water by spray application at a temperature in the range from 55° C. to 110°;
(b) sequential or simultaneous application of a dissolved Pd-containing precursor compound and a dissolved Au-containing precursor compound by spray application onto the bed of the catalyst support subjected to the recirculating motion, wherein in the case of simultaneous application the temperature is the same as in step (a) and wherein in the case of sequential application the temperature of the first application is the same as in step (a).

It has surprisingly been found that the processes according to the invention make it possible to produce shell catalysts which feature improved Pd distribution and Au distribution in the interior of the individual catalyst shaped bodies, wherein the maximum of the Pd concentration and the maximum of the Au concentration are in each case disposed in a range from 0 to 40 micrometers from the surface of the catalyst shaped body and the distance between the maximum of the palladium concentration and the gold concentration is in the range from 0 to 10 micrometers.

In the context of the present invention a shell catalyst is to be understood as meaning a catalyst which is in the form of a Pd- and Au-containing catalyst shaped body and in which the Pd compound and Au compound are present in the outer region of the support material as the catalytically active species and form a so-called shell while the interior of the shaped body is substantially free from Pd compounds and Au compounds. The shell typically exhibits a high concentration of the catalytically active species in a narrow region of the catalyst shaped body.

With regard to the two processes according to the invention the shell catalyst is in the form of a catalyst shaped body after step (b); prior to this a catalyst support is concerned.

The maximum of the Pd concentration and the maximum of the Au concentration in the shell catalyst are in the range from 0 to 40 micrometers, preferably in the range from 5 to 40 micrometers, preferably 5 to 35 micrometers, particularly preferably 5 to 30 micrometers, from the geometric surface of the catalyst shaped body in each case.

The distance between the maximum of the palladium concentration and the maximum of the gold concentration is in the range from 0 to 10 micrometers, preferably in the range from 0 to 8 micrometers, particularly preferably in the range from 0 to 5 micrometers.

In one embodiment the maximum of the Pd concentration is closer to the geometric surface than the maximum of the Au concentration while in another embodiment the maximum of the Au concentration is closer to the geometric surface than the maximum of the Pd concentration. In one embodiment the maximum of the Pd concentration and the maximum of the Au concentration are at the same distance from the geometric surface. It is preferable when the maximum of the Pd concentration is at the same distance from the geometric surface as the maximum of the Au concentration or closer to the geometric surface than the maximum of the Au concentration.

The shell catalyst comprises a shell comprising Pd and Au having a thickness of 30 to 200 micrometers, preferably 40 to 180 micrometers, particularly preferably 50 to 160 micrometers, most preferably 60 to 140 micrometers.

Subjecting a bed of a catalyst support to a recirculating motion is typically performed using suitable fluidized bed apparatuses. Such apparatuses are marketed by Glatt GmbH (Binzen, Germany), Aeromatic-Fielder AG (Bubendorf, Switzerland), Fluid Air Inc. (Aurora, Illinois, USA), Hüttlin GmbH (Steinen, Germany), Umang Pharmatech Pvt. Ltd. (Marharashtra, India) and Romaco Pharmatechnik GmbH (Karlsruhe, Germany). Fluidized bed apparatuses particularly preferred for performing the process according to the invention are marketed by Romaco Pharmatechnik GmbH under the trade names Innojet® Ventilus or Innojet® AirCoater. These apparatuses comprise a cylindrical vessel with a fixed and immovably installed vessel bottom, in the center of which a spray nozzle is mounted. The bottom is composed of circular slats mounted one above the other in steps. The process air flows into the vessel between the individual slats horizontally and eccentrically with a circumferential flow component outward in the direction of the vessel wall. This forms so-called sliding air layers on which the shaped catalyst support bodies are initially transported outward in the direction of the vessel wall. On the outside at the vessel wall is installed a vertically aligned process air stream which deflects the catalyst supports upward. Having arrived at the top, the catalyst supports move on a tangential path back towards the center of the base, in the course of which they pass through the spray mist of the nozzle. After passing through the spray mist the described motion begins again. The described process air management provides the basis for a substantially homogeneous toroidal fluidized bed-like recirculating motion of the catalyst supports.

In the processes according to the invention it is preferable to generate a fluidized bed in which the recirculating shaped bodies follow an elliptical or toroidal path. In the prior art the transition of the particles of a bed into a state in which the particles become entirely freely movable (fluidized bed) is referred to as the minimum fluidization point and the corresponding fluid velocity is referred to as the minimum fluidization velocity. It is preferable according to the invention when in the processes according to the invention the fluid velocity is up to 4 times the minimum fluidization velocity, preferably up to 3 times the minimum fluidization velocity and more preferably up to 2 times the minimum fluidization velocity.

In the processes according to the invention the catalyst supports recirculate in the fluidized bed along an elliptical or toroidal path, preferably a toroidal path. According to the invention “elliptical recirculation” is to be understood as meaning that the catalyst supports in the fluidized bed follow an elliptical path with varying size of the primary and secondary axis in the vertical plane. In the case of “toroidal recirculation” the catalyst supports in the fluidized bed follow an elliptical path with varying size of the primary and secondary axis in the vertical plane and a circular path with varying size of the radius in the horizontal plane. On average in the case of “elliptical recirculation” the catalyst supports follow an elliptical path in the vertical plane and in the case of “toroidal recirculation” follow a toroidal path, i.e. a catalyst support travels helically along the surface of a torus with a vertically elliptical section.

In one embodiment an acetate is applied to the catalyst supports before application of the Pd- and Au-containing precursor compounds. This is preferably done using a solution containing the acetate. The employed solvent for producing the solution is preferably water, more preferably deionized water. The acetate employed is typically an alkali metal acetate, preferably potassium acetate.

The application of the solution containing the acetate may be performed by any process known in the prior art, for example wet-chemical impregnation, the pore filling method (incipient wetness) and by spray impregnation of any type.

The application of the acetate is preferably carried out before step a) of the process according to the invention.

If the acetate is in the form of alkali metal acetate the loading of alkali metal is in the appropriate stoichiometric ratio to the acetate anions.

After the application of the acetate-containing solution it is preferable to perform a drying in the temperature range from 70° C. to 120° C., more preferably 80° C. to 110° C. and most preferably 90° C. to 100° C. in air, lean air or inert gas. The duration for drying of the acetate-laden support bodies is preferably in the range from 10 to 100 minutes, more preferably 30 to 60 minutes. The drying of the acetate-laden support bodies may be performed in a conventional drying apparatus but also in the coating apparatus. If the drying is performed in the coating apparatus this is preferably done such that the support bodies are in a static state therein, i.e. that they are set into motion. In case of using a moving bed or fluidized bed apparatus the support bodies are preferably not set into motion in the moving bed or the fluidized bed during the drying.

In step (b) a dissolved Pd precursor compound and a dissolved Au precursor compound are applied to the bed of the catalyst support subjected to the recirculating motion.

Examples of preferred Pd precursor compounds are water-soluble Pd salts. Particular preference is given to the Pd precursor compound selected from the group consisting of Pd(NH₃)₄(HCO₃)₂, Pd(NH₃)₄(HPO₄), ammonium Pd oxalate, Pd oxalate, K₂Pd(oxalate)₂, Pd(II) trifluoroacetate, Pd(NH₃)₄(OH)₂, Pd(NO₃)₂, H₂Pd(OAc)₂(OH)₂, freshly precipitated Pd(OH)₂.

If freshly precipitated Pd(OH)₂ is used it is preferably produced as follows: A 0.1% to 40% by weight aqueous solution is preferably produced from tetrachloropalladate. A base is then preferably added to this solution until a brown solid, namely the Pd(OH)₂ precipitates. To produce a solution for applying to the catalyst support the freshly precipitated Pd(OH)₂ is isolated, washed and dissolved in an aqueous alkaline solution.

The compound Pd(NH₃)₄(OH)₂ is preferably produced as follows: A precursor compound such as for example Na₂PdCl₄ is—as described above—precipitated with potassium hydroxide to afford palladium hydroxide and the precipitate, after filtration and washing, dissolved in aqueous ammonia to afford Pd(NH₃)₄(OH)₂.

The process according to the invention may also employ Pd nitrite compounds. Preferred Pd nitrite precursor compounds are for example those obtained by dissolving Pd(OAc)₂ in an NaNO₂ or KNO₂ solution.

Examples of preferred Au precursor compounds are water-soluble Au salts. According to a particularly preferred embodiment of the process according to the invention the Au precursor compound is selected from the group consisting of KAuO₂, NaAuO₂, CsAuO₂, NMe₄AuO₂, KAUCl₄, (NH₄)AuCl₄, HAuCl₄, KAu(NO₂)₄, NaAu(NO₂)₄, CsAu(NO₂)₄, AuCl₃, NaAuCl₄, CsAuCl₄, KAu(OAc)₃(OH), NaAu(OAc)₃(OH), CsAu(OAc)₃(OH), HAu(NO₃)₄ and Au(OAc)₃. It may be advantageous to freshly prepare the Au(OAc)₃ or the KAuO₂ by precipitation of the oxide/hydroxide from an auric acid solution, washing and isolation of the precipitate and dissolution thereof in acetic acid/KOH.

In the context of the present invention dissolved Pd-containing precursor compounds and Au-containing precursor compounds are to be understood as compounds which are present in dissolved form in a solvent, preferably water, aqueous base or aqueous acid.

In the context of the present invention salts are described as water-soluble if they are sufficiently soluble in water, aqueous base or aqueous acid.

It is preferable when the Pd- and Au-precursor compounds are selected such that they contain no catalyst poisons and after application of the noble metal compounds the catalyst shaped bodies are merely dried, preferably in the same fluidized bed coater as application was carried out in. The precursor compounds are particularly preferably substantially chloride-free. Substantially chloride-free is to be understood as meaning that the molecular formula of the compound contains no chloride though this does not exclude the possibility that the compound contains unavoidable impurities of chloride as a consequence of production for example. The content of chloride in substantially chloride-free Pd and Au precursor compounds is at most 125 ppm per % by weight of Pd and Au in the precursor compound.

The application of the Pd-containing precursor compound and the Au-containing precursor compound is carried out by sequential, i.e. successive, or simultaneous application of a dissolved Pd-containing precursor compound and a dissolved Au-containing precursor compound by spray application onto the bed of the catalyst support subjected to the recirculating motion. This application may be effected by simultaneous application of a solution of a Pd-containing precursor compound and a solution of an Au-containing precursor compound. It may also be effected by simultaneous application of a solution of a Pd-containing precursor compound and an Au-containing precursor compound. It may also be effected by sequential application, wherein in a first step a solution of a Pd-containing precursor compound and in a second step a solution of an Au-containing precursor compound is effected or in a first step a solution of an Au-containing precursor compound and in a second step a solution of a Pd-containing precursor compound is effected.

In one embodiment the solution applied in the second step in the case of sequential application contains both a Pd-containing precursor compound and an Au-containing precursor compound.

If the shell catalyst is to contain a plurality of distinct catalytically active species in the shell, for example a plurality of active metals or an active metal and a promoter metal, the catalyst support shaped body may be subjected to the process according to the invention a corresponding number of times. Alternatively the process according to the invention may also be performed with mixed solutions which contain the desired distinct catalytically active species or precursors thereof. The catalyst supports may moreover be simultaneously sprayed with distinct solutions of catalytically active species or precursors thereof in the process according to the invention.

The process gas used in the process according to the invention is typically air. In a further embodiment it is also possible to employ an inert gas, for example nitrogen, methane, short-chain saturated hydrocarbons, a natural gas, preferably helium, neon or argon, or a halogenated hydrocarbon or a mixture of two or more of these.

Targeted heating of the catalyst support in step (a) and in step b) of the first process according to the invention makes it possible to control the width of the noble metal shell formed since depending on temperature in the case of combined application of the Pd-containing precursor compound and Au-containing precursor compound faster evaporation of the at least one preferably aqueous solution applied in step b) occurs or in the case of sequential application of the Pd-containing precursor compound and Au-containing precursor compound faster evaporation of the first-applied preferably aqueous solution applied in step b) occurs and diffusion of the noble metals into the carrier interior is thus reduced. At relatively high temperatures for example the drying rate is relatively high so that solution contacting the catalyst support dries virtually immediately, as a result of which a solution applied to the catalyst support cannot penetrate deeply into said support. At relatively high temperatures it is thus possible to obtain shells having a relatively low thickness and a high loading of active species.

It has now been found that, surprisingly, in the first process according to the invention the heating of the catalyst support carried out in step (a) to a temperature which is 5° C. to 30° C., preferably 7° C. to 25° C., most preferably 10° C. to 20° C., above the temperature of application or in the case of sequential application the temperature of the first application in step (b) makes it possible to advantageously control the distribution of the Pd and Au such that the maximum of the Pd concentration and the maximum of the Au concentration are in a range from 0 to 40 micrometers from the surface of the catalyst shaped body and the distance between the maximum of the palladium concentration and the gold concentration is in the range from 0 to 10 micrometers.

The temperature of the catalyst support to be established in step (a)/(b) is controlled by measuring the exhaust air temperature/temperature of the bed and optionally effecting follow-up control.

The temperature to be established in step (a) is in the range from 60° C. to 120° C., preferably of 65° C. to 110° C., particularly preferably of 70° C. to 100° C.

The temperature of application or in the case of sequential application the temperature of the first application to be established in step (b) is in the range from 55° C. to 115° C., preferably in the range from 55° C. to 110° C., more preferably in the range from 60° C. to 100° C., particularly preferably in the range from 65° C. to 90° C. In the case of sequential application the second application may be carried out at the same temperature as the first application. In one embodiment the temperature during the second application is 5° C. to 30° C., preferably 5° C. to 20° C., most preferably 5° C. to 10° C., higher than during the first application. The spraying with water in step a) of the second process according to the invention makes it possible to control the width of the noble metal shell formed to the same extent since the water evaporates during the contacting with the catalyst support and results in a transient temperature reduction of the catalyst support which is re-compensated by the temperature control of the coating apparatus so that the subsequent application of the metal-containing aqueous solutions may be effected at the desired target temperature, thus making it possible to achieve a faster evaporation of the aqueous solution applied in step b) or in the case of sequential application faster evaporation of the first applied solution and thus reducing diffusion of the noble metals into the support interior. At relatively high temperatures for example the drying rate is relatively high so that solution contacting the catalyst support dries virtually immediately, as a result of which a solution applied to the catalyst support cannot penetrate deeply into said support. At relatively high temperatures it is thus possible to obtain shells having a relatively low thickness and a high loading of active species.

The temperature of the catalyst support to be established in step (a)/(b) is controlled by measuring the exhaust air temperature/temperature of the bed and optionally effecting follow-up control.

The temperature to be established in step (a) is in the range from 55° C. to 110° C., preferably of 60° C. to 100° C., particularly preferably of 65° C. to 90° C.

The temperature of application or in the case of sequential application the temperature of the first application to be established in step (b) is the same as in step (a) and is in the range from 55° C. to 110° C., preferably from 60° C. to 100° C., particularly preferably 65° C. to 90° C. In the case of sequential application the second application may be carried out at the same temperature as the first application. In one embodiment the temperature during the second application is 5° C. to 30° C., preferably 5° C. to 20° C., most preferably 5° C. to 10° C. higher.

In the context of the process according to the invention the drying of the catalyst support sprayed with the solution is preferably carried out continuously using the process gas.

In one embodiment of the processes according to the invention the catalyst support is subjected to a fixation step after step (b) to fix the catalytically active species or their precursors on the catalyst support. Suitable measures include spraying the catalyst supports laden with the noble metal compounds with a lye or immersing the catalyst supports in a lye-containing solution. This is followed by a treatment with water to remove excess lye from the catalyst support. This process is especially suitable when the applied Pd or optionally Au precursor compound contains constituents which act as catalyst poisons, in particular in the production of VAM, for example the corresponding Pd or Au chlorides. In a preferred embodiment the catalyst support obtained after step (b) is subjected to thermal treatment at a temperature in the range from 300° C. to 600° C., especially for 1 h to 6 h, to convert the precursor compounds into the corresponding hydroxide compounds/oxides. In a further embodiment the catalyst support obtained after step (b) is dried at a temperature in the range from 80° C. to 200° C., preferably 100° C. to 150° C.

In a further embodiment the catalyst support obtained after step (b) is not subjected to a fixation step but dried at a temperature in the range from 80° C. to 200° C., preferably 100° C. to 150° C.

However, it is particularly preferable when neither a step of drying nor the thermal treatment in the range from 300° C. and 600° C. is carried out after step (b) and before the reduction in a step (c).

In one embodiment step (b) is followed by a step (c) comprising reduction of the metal components of the precursor compounds to the elemental metals by subjecting the catalyst shaped body obtained after step (b) to a heat treatment in a non-oxidizing atmosphere.

The reduction in step (c) may be carried out in the fluidized bed coater itself or in a separate reduction reactor. If the reduction is carried out in the fluidized bed coater this is typically performed with a mixture of 28 to 5% by volume of hydrogen and nitrogen at a temperature in the range from 70° C. to 150° C. over a period of for example 30 min to 5 hours. If the reaction is performed in a separate reduction reactor this is typically performed with a mixture of 28 to 5% by volume of hydrogen in nitrogen, for example a forming gas, at temperatures in the range of preferably 70-500° C. over a period of 30 min to 5 hours.

Suitable further reducing agents include ethylene, CO, NH₃, formaldehyde, methanol and hydrocarbons, wherein the reducing agents may also be diluted with an inert gas, for example carbon dioxide, nitrogen or argon. It is preferable when a reducing agent diluted with inert gas is employed. It is preferable to employ mixtures of hydrogen with nitrogen or argon, preferably with a hydrogen content between 1% by volume and 15% by volume.

In a further embodiment the reduction of the noble metals in a pure nitrogen atmosphere may be performed at a temperature in the range from 130° C. to 200° C., preferably in the range from 140° C. to 170° C.

In a further embodiment the reduction of the noble metals may also be performed in the liquid phase, preferably using the reducing agents hydrazine, potassium formate, sodium formate, formic acid, H₂O₂, hypophosphoric acid or Na hypophosphite.

The catalyst support used in the process according to the invention may have various shapes, for example tablets, cylinders, rings, irregular pellets or spheres. The catalyst support is preferably spherical. The geometry thereof makes it possible to achieve uniform rotation of the support about its axis in the recirculating motion, thus resulting in uniform impregnation of the catalyst support with the solution of the catalytically active species. The catalyst support used in the process according to the invention is not a pulverulent material.

The spherical catalyst support has an arithmetic diameter in the range from 1 to 10 mm, preferably 3 to 9, particularly preferably 3 to 8 mm. In the context of the present application the term “spherical” is also to be understood including a spheroid body, wherein the diameter along the axis from pole to pole is smaller than the diameter along the corresponding equatorial axis. The ratio between the diameter along the axis from pole to pole and the diameter along the equatorial axis is in the range from 0.9 to 1.0. The arithmetic diameter of these very largely spherical bodies is determined using the diameter from pole to pole.

The catalyst support typically comprises a compound from the group of titanium oxide, silicon oxide, aluminum oxide, zirconium oxide, magnesium oxide, silicon carbide, magnesium silicate, zinc oxide, zeolites, layered silicates or mixtures thereof. It is preferable when the catalyst support comprises a Si—Al mixed oxide, in particular in the form of a layered silicate, preferably a layered silicate in the form of a calcined acid-treated bentonite. The proportion of these compounds or their mixtures is typically at least 70% by weight, preferably at least 80% by weight, particularly preferably at least 90% by weight, based on the weight of the catalyst support after loss on ignition. In a particularly preferred embodiment the catalyst support comprises a calcined acid-treated bentonite in a proportion of at least 70% by weight, preferably at least 80% by weight, particularly preferably at least 90% by weight and most preferably at least 95% by weight based on the weight of the catalyst support after loss on ignition.

The term “natural layered silicate”, synonymous in the literature with the term “phyllosilicate”, is understood to mean untreated or treated silicate mineral derived from natural sources in which SiO₄ tetrahedrons are crosslinked with one another in layers of general formula [Si₂O₅]²⁻. These tetrahedron layers alternate with so-called octahedron layers in which a cation, especially Al and Mg, is octahedrally surrounded by OH or O. A distinction is made between bilayer phyllosilicates and trilayer phyllosilicates for example. Layered silicates preferred in the context of the present invention include clay minerals, in particular kaolinite, beidellite, hectorite, saponite, nontronite, mica, vermiculite and smectites, wherein smectites and in particular montmorillonite are particularly preferred. Definitions of the term “layered silicates” may be found for example in “Lehrbuch der anorganischen Chemie”, Hollemann Wiberg, de Gruyter, 102nd edition, 2007 (ISBN 978-3-11-017770-1) or in “Römpp Lexikon Chemie”, 10th edition, Georg Thieme Verlag under the term “phyllosilicate”. Typical treatments to which a natural phyllosilicate is subjected before use as support material include for example treatment with acids and/or calcining. A natural particularly preferred layered silicate in the context of the present invention is a bentonite. Bentonites are not natural layered silicates in the strict sense but rather a mixture of predominantly clay minerals which contain layered silicates. In the case where the natural layered silicate is a bentonite it is to be understood that the natural layered silicate is present in the catalyst support in the form of or as a constituent of a calcined acid-treated bentonite.

A catalyst support in the form of a shaped body and based on natural layered silicates, in particular based on an acid-treated calcined bentonite, may be produced for example by shaping an acid-treated (uncalcined) bentonite and water-containing mixture by compression to afford a shaped body using apparatuses familiar to those skilled in the art, for example extruders or tablet presses, and subsequently calcining the uncured shaped body to afford a stable shaped body. The size of the specific surface area of the catalyst support depends in particular on the quality of the employed (raw) bentonite, the acid treatment process of the employed bentonite, i.e. for example the nature and the amount relative to the bentonite and concentration of the employed inorganic acid, the acid treatment duration and the temperature, on the pressing pressure and on the calcining duration and temperature and the calcining atmosphere.

Acid-treated bentonites are obtainable by treatment of bentonites with strong acids, for example sulfuric acid, phosphoric acid or hydrochloric acid. A definition of the term bentonite which also applies in the context of the present invention is specified in Römpp, Lexikon Chemie, 10th edn., Georg Thieme Verlag. Bentonites particularly preferred in the context of the present invention are natural aluminum-containing layered silicates containing montmorillonite as the main material. After acid treatment the bentonite is generally washed with water, dried and ground to a powder.

The BET surface area of the catalyst support is 10 to 600 m²/g, preferably 20 to 400 m²/g and particularly preferably between 80 and 170 m²/g. The BET surface area is determined by the 1-point method by adsorption of nitrogen according to DIN 66132.

In one embodiment the integral pore volume of the catalyst support (determined according to DIN 66133 (Hg porosimetry)) without the coating with the precursor compound is at least 0.1 ml/g, preferably at least 0.18 ml/g, more preferably at least 0.4 ml/g. In a further embodiment the integral pore volume is in the range from 0.1 ml/g to 0.8 ml/g, preferably in the range from 0.18 ml/g to 0.6 ml/g and particularly preferably in the range from 0.35 to 0.55 ml/g.

The catalyst support used in the process according to the invention has a hardness of at least 20 N, preferably of at least 30 m, particularly preferably of at least 40 N and most preferably of at least 50 N.

The proportion of Pd in the catalyst according to the invention is in the range from 0.2% to 2.0% by weight, preferably in the range from 0.4% to 1.75% by weight and particularly preferably in the range from 0.7% to 1.5% by weight based on the mass of the catalyst shaped body laden with noble metal and reduced after loss on drying.

The proportion of Au in the catalyst according to the invention is in the range from 0.1% to 1.2% by weight, preferably in the range from 0.2% to 1.0% by weight and most preferably in the range from 0.3% to 0.8% by weight based on the total weight of the catalyst shaped body after reduction and after loss on drying.

In a preferred embodiment the Au/Pd atom ratio of the catalyst shaped body according to the invention is in the range from 0.01 to 1.2, preferably in the range from 0.05 to 1.0, more preferably in the range from 0.1 to 0.8 and particularly preferably in the range from 0.15 to 0.6.

The shell catalyst according to the invention preferably contains an alkali metal compound as a promoter, preferably a potassium, a sodium, a cesium or a rubidium compound, particularly preferably a potassium compound. Suitable and particularly preferred potassium compounds include potassium acetate KOAc, potassium carbonate K₂CO₃, potassium hydrogen carbonate KHCO₃ and potassium hydroxide KOH, preferably potassium acetate KOAc. The potassium compound may be applied to the catalyst support/catalyst shaped body either before or after reduction of the metal components to the metals Pd and Au. In a preferred embodiment the potassium compound is applied before application of the noble metal compounds, particularly preferably before step (a). In this case care must be taken to ensure that in the subsequent steps such as application of the precursor compounds, a possible fixation or the reduction a thermal treatment is carried out only insofar as this does not result or results only to a small extent in the decomposition of the potassium compound into the corresponding oxide since the potassium oxide is converted into potassium acetate only insufficiently under the reaction conditions of the VAM reaction. It is preferable when the catalyst support/catalyst shaped body is exposed only to temperatures below 200° C.

If the catalyst shaped body according to the invention comprises an alkali metal acetate, preferably potassium acetate, the content of the alkali metal acetate in the catalyst bed is 0.1 to 0.7 mol/l, preferably 0.3 to 0.5 mol/l.

In a preferred embodiment of the catalyst shaped body according to the invention the alkali metal/Pd atom ratio is in the range from 1 to 16, more preferably in the range from 2 to 13 and particularly preferably in the range from 3 to 10. Preferably, the lower the alkali metal/Pd atom ratio, the smaller the surface area of the catalyst shaped body.

Having regard to a low pore diffusion limitation it may be provided in a further preferred embodiment of the Pd/Au catalyst according to the invention that the catalyst shaped body has an average pore diameter of 8 to 50 nm, by preference 10 to 35 mm and preferably 11 to 30 nm.

The acidity of the catalyst shaped body can have an advantageous effect on the activity of the catalyst according to the invention. In a further preferred embodiment of the catalyst according to the invention the catalyst shaped body has an acidity in the range from 1 to 150 μval/g, preferably in the range from 5 to 130 μval/g and particularly preferably in the range from 10 to 100 μval/g. The acidity of the catalyst shaped bodies is determined as follows: 1 g of the finely ground catalyst shaped body is admixed with 100 ml of water (having a pH blank value) and extracted for 15 minutes with stirring. The mixture is subsequently titrated at least to pH 7.0 with 0.01 n NaOH solution, wherein the titration is carried out in stages comprising; initially adding 1 ml of the NaOH solution to the extract dropwise (1 droplet/second), then waiting for 2 minutes, reading off the pH, adding a further 1 ml of NaOH dropwise etc. The blind value of the employed water is determined and the acidity calculation corrected accordingly.

The titration curve (ml 0.01 NaOH vs. pH) is then plotted and the intersection point of the titration curve at pH 7 is determined. The mole equivalents are calculated in 10-6 equiv/g support and result from the NaOH consumption for the intersection point at pH 7.

$$Total acid : \frac{10 \cdot ml 0.01 n NaOH}{catalyst support} = μ val / g .$$

The shell catalyst is preferably spherical or virtually spherical. The sphere has a diameter in the range from 1 to 10 mm, preferably 3 to 9 mm, particularly preferably 3 to 8 mm.

To increase the activity of the Pd/Au catalyst according to the invention it may be provided that the catalyst shaped body is doped with at least one oxide of a metal selected from the group consisting of Zr, Hf, Ti, Nb, Ta, W, Mg, Re, Y and Fe, preferably with ZrO₂, HfO₂ or Fe₂O₃. It may be preferable when the doping oxide proportion of the catalyst shaped body is between 0.1% and 20% by mass, preferably 1.0% to 18% by mass and preferably 4% to 16% by mass based on the mass of the catalyst shaped body.

In a further preferred embodiment of the catalyst shaped body according to the invention it may be provided that the water absorbency of the catalyst shaped body is 40% to 758, preferably 50% to 70%, calculated as weight gain by water absorption. The absorbency is determined by impregnating 10 g of the catalyst shaped body with deionized water for 30 min until no more gas bubbles escape from the sample. The excess water is then decanted and the impregnated sample is dabbed dry with a cotton cloth to free the sample of adherent moisture. The water-laden catalyst shaped body is then weighed out and the absorbency calculated according to:

$$Final weight ( g ) - initial weight ( g ) ) \times 10 = water absorption capacity ( \% )$$

The BET surface area of the shell catalyst is 10 to 600 m²/g, preferably 20 to 400 m²/g and particularly preferably between 80 and 170 m²/g. The BET surface area is determined by the 1-point method by adsorption of nitrogen according to DIN 66132.

In one embodiment the integral pore volume of the shell catalyst (determined according to DIN 66133 (Hg porosimetry)) without the coating with the precursor compound is at least 0.1 ml/g, preferably at least 0.18 ml/g, more preferably at least 0.4 ml/g. In a further embodiment the integral pore volume is in the range from 0.1 ml/g to 0.8 ml/g, preferably in the range from 0.18 ml/g to 0.6 ml/g and particularly preferably in the range from 0.35 to 0.55 ml/g.

The shell catalyst has a hardness of at least 20 N, preferably of at least 30 N, particularly preferably of at least 40 N and most preferably of at least 50 N.

In a further preferred embodiment of the catalyst shaped body according to the invention it may be preferable when at least 80% of the integral pore volume of the catalyst shaped body is formed by mesopores and macropores, by preference at least 85% and preferably at least 90%. This counters a reduced activity of the catalyst shaped body according to the invention resulting from diffusion limitation, in particular in the case of shells with relatively large thicknesses. The terms micropores, mesopores and macropores are to be understood as meaning pores having a diameter of less than 2 nm, a diameter in the range from 2 to 50 nm and a diameter of greater than 50 nm respectively.

The present invention further relates to a process for producing alkenylcarboxylic esters, in particular VAM or allyl acetate monomer with the shell catalyst according to the invention. The process for producing VAM is performed by passing acetic acid, ethylene and oxygen or oxygen-containing gases over the catalyst shaped body according to the invention. This is generally done by passing gases containing acetic acid, ethylene and oxygen or oxygen-containing gases over the catalyst shaped body according to the invention at temperatures in the range from 100° C. to 200° C., preferably in the range from 120° C. to 200° C., and at pressures in the range from 1 to 25 bar, preferably in the range from 1 to 20 bar, wherein unconverted reactants may be recirculated. The oxygen concentration is advantageously kept below 10% by volume. However, dilution with inert gases such as nitrogen or carbon dioxide may also be advantageous. Carbon dioxide is particularly suitable for dilution since it is formed in small quantities in the course of the VAM synthesis and accumulates in the circulating gas. The resulting vinyl acetate is isolated using suitable methods which are described for example in U.S. Pat. No. 5,066,365 A. Equivalent processes have also been published for allyl acetate. It is also known that in such processes for producing vinyl acetate it is possible to carry out post-addition of promoters which may be present in the catalyst, for example potassium acetate KOAc, to resupply proportions of promoter lost during the service life of the catalysts in the process. This may be carried out during ongoing operation of the process for producing alkenylcarboxylic esters such that acetate compounds of the promoters are admixed with the acetic acid to be supplied to the process and/or the potassium acetate to be supplied to the process or are metered in separately.

The invention shall now be more particularly elucidated with reference to a plurality of working examples though these shall not be considered limiting.

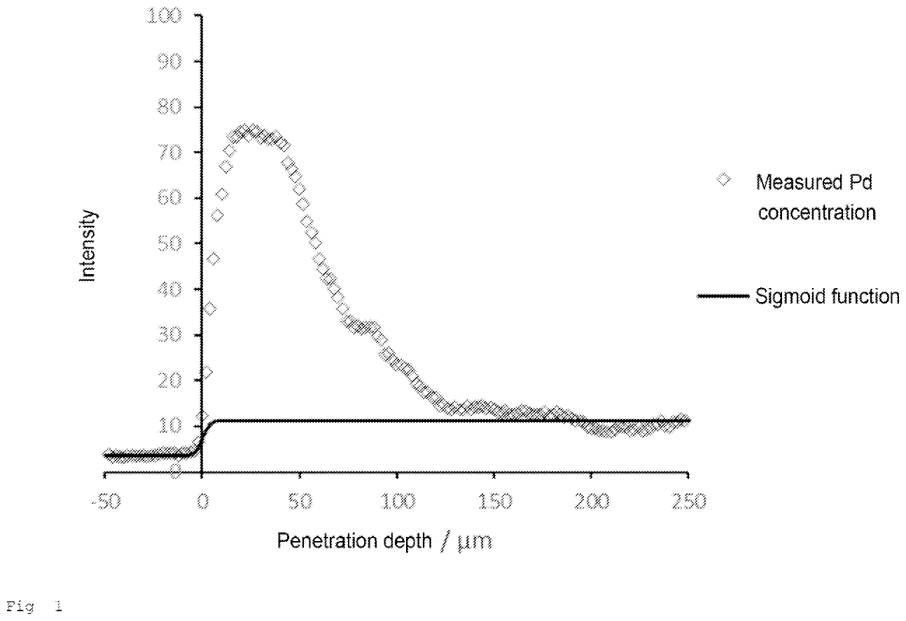

FIG. 1 shows by way of example for catalyst 1 a plot of the experimental data points of the Pd concentrations and the sigmoid function determined by means of the same measurement for determining the start of the geometric surface after determination of x_{surface}.

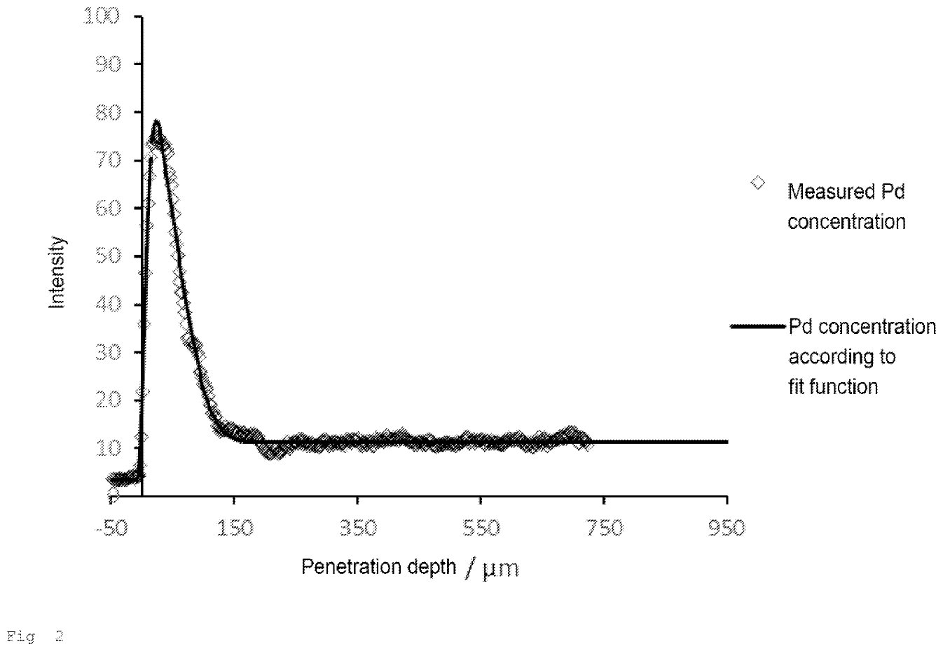

FIG. 2 shows a plot of the experimental data points of the Pd concentrations and the curve of catalyst 1 obtained using the appropriate fit function.

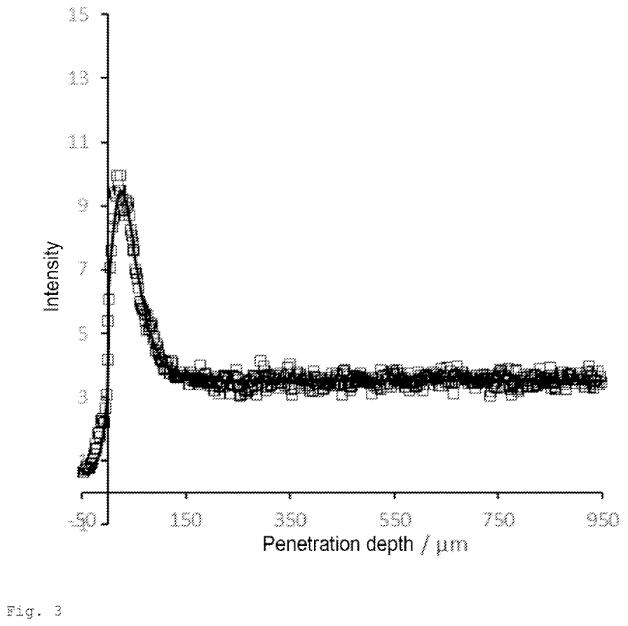

FIG. 3 shows a plot of the experimental data points of the Au concentrations and the curve of catalyst 1 obtained using the appropriate fit function.

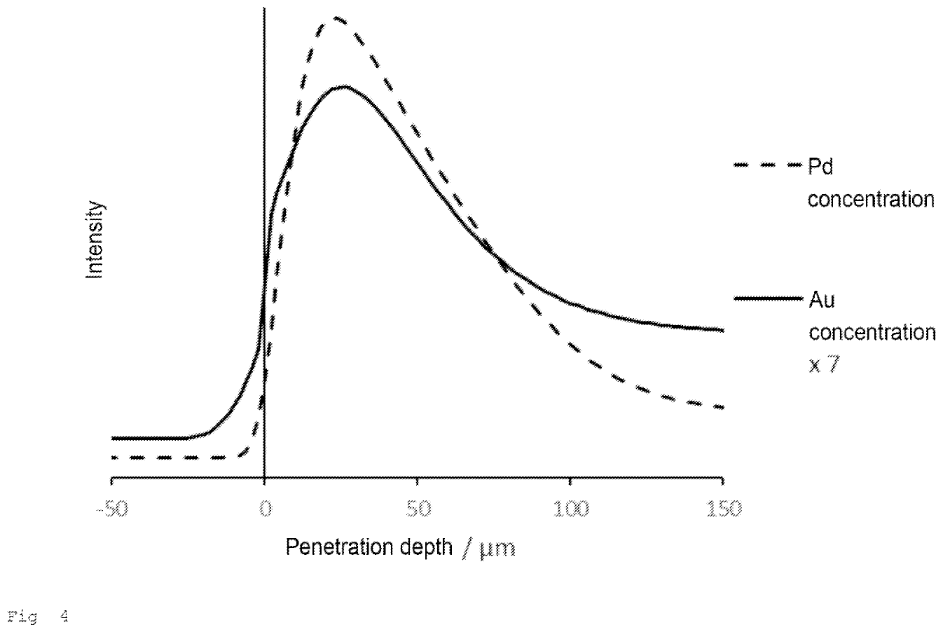

FIG. 4 shows a plot of the curves of the Pd concentrations and the Au concentrations of catalyst 1 obtained using the appropriate fit functions.

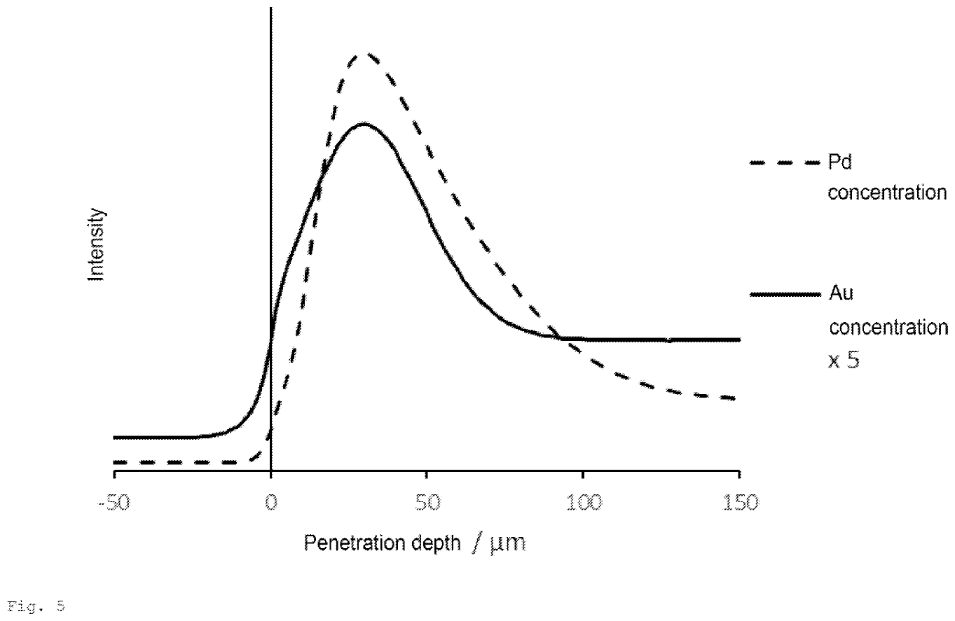

FIG. 5 shows a plot of the curves of the Pd concentrations and the Au concentrations of catalyst 2 obtained using the appropriate fit functions.

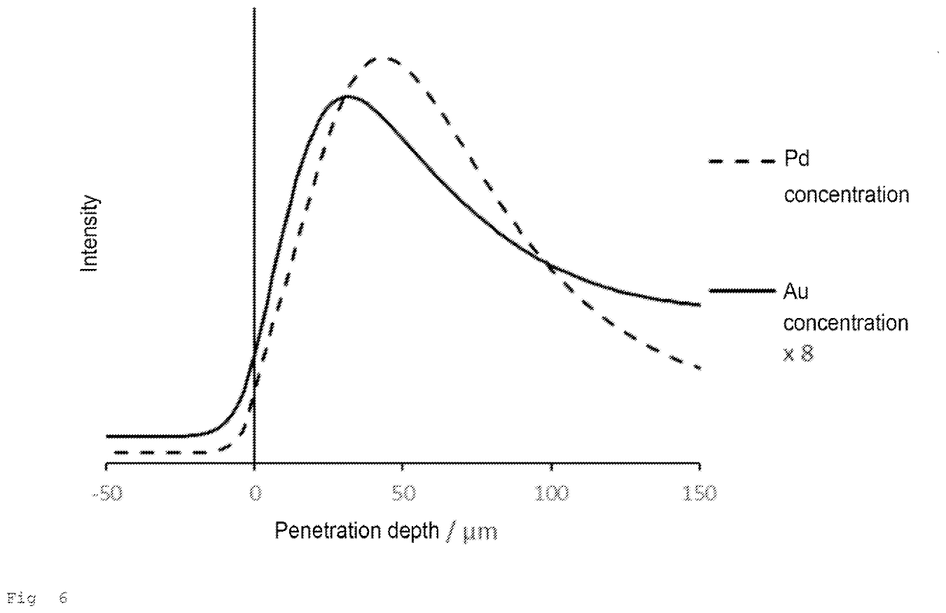

FIG. 6 shows a plot of the curves of the Pd concentrations and the Au concentrations of catalyst 3 obtained using the appropriate fit functions.

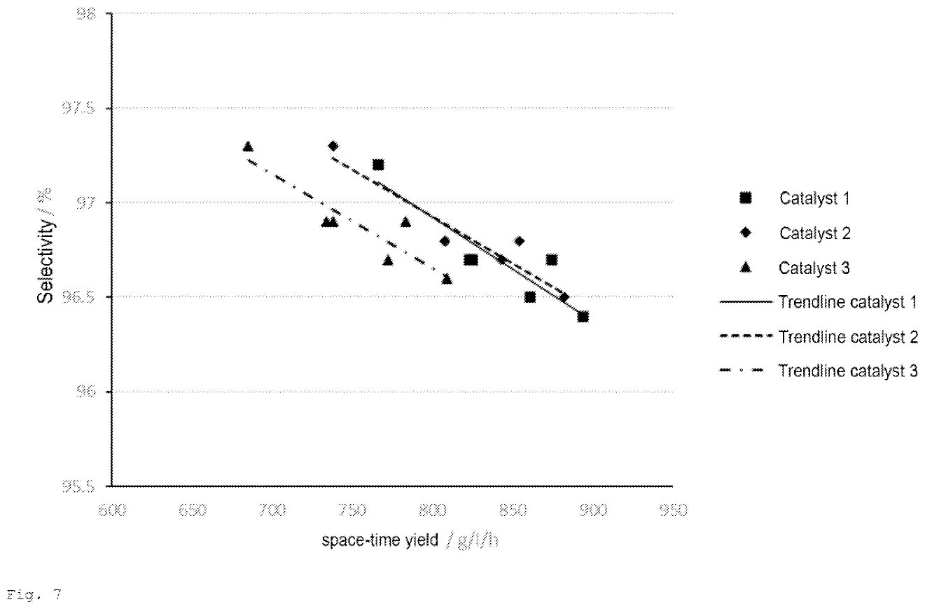

FIG. 7 shows a plot of the selectivities at different space-time yields of the catalysts 1 and 2 and their associated trend lines in the reaction of acetic acid, ethylene and oxygen to afford vinyl acetate monomer.

### WORKING EXAMPLES

### Methods of Measurement

### Pore Volume

Pore volume was measured by the mercury porosimetry method according to DIN 66133 in a pressure range from 1 to 2000 bar.

### BET Surface Area

BET surface areas were determined according to DIN 66135. To determine micropore volume and micropore surface area the adsorption isotherms of nitrogen at the temperature of liquid nitrogen (77 K) was determined with a Micromeritics ASAP 2020 M instrument.

### Element Distribution in Support Material

The distribution of the Pd and Au in the catalyst shaped body was determined by making a section of the spherical catalyst shaped body by halving the support. This made it possible to determine the spatial distribution of the metal using EDX spectroscopy (energy dispersive X-ray diffraction), also known as EDX spectroscopy (Energy Dispersive X-ray), with the electron microscope. A measuring head which is sensitive to Pd and Au was passed across the sample, thus making it possible to determine the respective distributions thereof along a line from the outer surface towards the middle of the catalyst shaped body.

Measurement was carried out using a LEO 1530 electron microscope coupled to a Quantax EDX unit with a a Bruker XFlash 4010 detector. Measurement conditions were as follows:
Acceleration voltage (EHT): 20 KV
Aperture: 120 μm
Working distance: about 16 mm (Optimum for SEM-EDX geometry)
SEM Detector: SE2
Magnification: Low (e.g. 55×)
Measurement duration: about 10-15 min.

The starting point of the measurement was set at least 80 μm from the outer edge of the sample and the measurement was performed from there over the sample toward the center of the sphere. The line of measurement had a length of 1600 μm.

The measurement for various catalyst geometries is generally carried out such that the starting point of the measurement is set at least 80 μm from the original geometric surface of the sample, the sample head is moved from there vertically in the direction of the original geometric surface of the sample and also further over the sample.

This made it possible to determine the shell thickness of the Pd and the shell thickness of the Au. The inner end of the shell thickness was defined as the point at which the intensity first has a value less than the sum of the braking radiation and three times the standard deviation thereof.

The start of the geometric surface of the measured catalyst shaped body was determined by fitting the function to the intensities of the braking radiation inside and outside the catalyst shaped body along the measured line at various points

$$I ( x ) = base + \frac{max}{1 + {e}^{\frac{{x}_{0} - x}{rate}}}$$

x. Rate is a measure for the gradient at the inflection point, base corresponds to the intensity I of the braking radiation outside the catalyst shaped body and max corresponds to the intensity I of the braking radiation inside the catalyst shaped body for the region without noble metal loading, i.e. outside the noble metal shell region. The inflection point x₀ of this sigmoid function defines the outer end of the shell and for the subsequent determination of the concentration distribution of the noble metals along the measured line this value x₀ was subtracted from the measured x-values so that all measurement series begin with the point x_{surface}=0 μm of the outer surface. FIG. 1 shows by way of example for catalyst 1 a graphical representation of the measured intensities and the sigmoid function determined by means of the same measurement for determining the start of the geometric surface after determination of x_{surface}.

This ensures that a sufficient number of individual catalyst shaped bodies are measured and have their outer surface determined.

The concentration distribution of the noble metals along the line was determined by calculating the arithmetic average of the intensities at point x from the individual measurements and, from the measured values resulting therefrom, performing a fitting of the concentration distribution of the noble metals using the following Gaussian function.

$$I ( x ) = A \times {e}^{- \frac{x - {x}_{max}}{max} \times ( 1 + {e}^{\frac{{x}_{max} - x}{rate}} )}$$

xₘₐₓ=position of the peak maximum
A=determines peak height
max=determines asymmetry of the concentration distribution
rate=determines the asymmetry of the concentration distribution

### Determination of Elemental Concentrations

The concentration of the elements was determined by dokimastic digestion via copper cupellation and subsequent ICP-AES analysis and was calculated based on the catalyst shaped body after drying for 2 h at 120° C.

### Determination of Hardness

The hardness of the catalyst shaped body was determined as an average on 99 catalyst shaped bodies using a Dr. Schleuniger Pharmatron AG 8M tablet hardness tester. The catalyst shaped bodies were dried for 2 h at 130° C. before measurement. The instrument settings were as follows:
Hardness: N
Distance to catalyst shaped body: 5.00 mm
Time delay: 0.80 sec
Feed type: 6 D
Speed: 0.60 mm/s

The following working examples are for elucidation of the invention.

### Example 1: Catalyst 1

100 g of the bentonite-containing catalyst support material KA-160 (available from Clariant) were weighed in and impregnated with a mixture of 39.3 g of 2 molar KOAc solution and 18.1 g of deionized water according to the pore filling method (incipient wetness). After static drying in the fluidized bed dryer at 90° C. for 35 min the mixture was cooled to room temperature and transferred to an Innojet IAC 025 coater. A process gas was used to set the KOAc-impregnated support into a fluidized bed motion. 36.8 g of deionized water were then sprayed onto the catalyst support at a spray rate rate of 4 g/min. The process air was temperature-controlled to a temperature of 70° C. Subsequently, 7.0 g of an aqueous caesium aurate solution (4.7% by weight Au) was diluted with deionized water to afford 50 g of coating solution and said coating was applied to the catalyst supports in a first coating step at a spray rate of 4 g/min and a process air temperature of 70° C. in the coater. Subsequently in a second coating step a mixture of 2.7 g of an aqueous cesium aurate solution (4.7% by weight Au) and 38.9 g of a tetraammine palladium dihydroxide solution (3.4% by weight Pd) was diluted with deionized water to afford 80 g of coating solution and applied to the catalyst supports at a spray rate of 4 g/min and a process air temperature of 70° C. The catalyst supports continue to be kept in a fluidized bed. After a further static drying in the fluidized bed dryer (90° C./35 min) the catalyst shaped body was subjected to static reduction in a tubular furnace with forming gas (2% H₂ in N₂) at 100° C. for 45 min.

The elemental analysis of the catalyst shaped body showed the following proportions:
Pd: 1.3% by weight
Au: 0.46% by weight

Determination of the noble metal distribution was carried out using a scanning electron microscope LEO 1530 provided with an energy dispersive spectrometer from Bruker AXS. To measure the noble metal concentration across the shell thickness a catalyst shaped body was dissected, glued to an aluminum sample holder and subsequently subjected to vapor deposition of carbon. The detector employed was a nitrogen-free silicon drift chamber detector (XFlash® 410) with an energy resolution of 125 eV for the manganese K-alpha line.

The following parameters were used for the measurement:
Scan resolution: 500 dots
Spacing of data points: 1.8 μm
Magnification: 200 times
Beam voltage 20 kV
Beam current 20 nA
Input pulse rate: 50 000 pulses/s.
Measurement time for line scan: 200 s

The shell thickness of 10 spheres of the shell catalyst batch produced as described above was measured.

The maximum of the Pd concentration was at 23 micrometers and the maximum of the Au concentration was 25 micrometers below the geometric surface of the shell catalyst. The shell thickness for Pd was 130 micrometers and for Au was 93 micrometers. The curve of the Pd concentrations based on the measured values as well as the values obtained by the corresponding fit function is shown in FIG. 2 and the curve of the Au concentrations based on the measured values as well as after adjustment by the corresponding fit function is shown in FIG. 3. FIG. 4 shows both curves of the fitted function, wherein for better comparability the values of the Au concentration have been multiplied by a factor of 7.

### Example 2: Catalyst 2

100 g of the bentonite-containing catalyst support material KA-160 (available from Clariant) were weighed in and impregnated with a mixture of 39.3 g of 2 molar KOAc solution and 18.1 g of deionized water according to the pore filling method (incipient wetness). After static drying in the fluidized bed dryer at 90° C. for 35 min the mixture was cooled to room temperature and transferred to an Innojet IAC 025 coater. A process gas was used to set the KOAc-impregnated support into a fluidized bed motion. The process air was temperature-controlled to a temperature of 90° C. and held for 2 minutes before commencement of spray-application of a cesium aurate solution. To this end 7.0 g of an aqueous cesium aurate solution (4.7% by weight Au) were diluted with deionized water to afford 50 g of coating solution and said solution was applied to the catalyst support in a first coating step at a spray rate of 4 g/min in the coater, the start of the spraying operation reducing the temperature of the process air by 15° C. from 90° C. and the process air further being actively reduced to 70° C. and then maintained at this temperature. Subsequently in a second coating step a mixture of 2.4 g of an aqueous cesium aurate solution (4.7% by weight Au) and 38.9 g of a tetraammine palladium dihydroxide solution (3.4% by weight Pd) was diluted with deionized water to afford 80 g of coating solution and applied to the catalyst supports at a spray rate of 4 g/min and a process air temperature of 70° C. The catalyst supports continue to be kept in a fluidized bed. After a further static drying in the fluidized bed dryer (90° C./35 min) the catalyst shaped body was subjected to static reduction in a tubular furnace with forming gas (2% H in No) at 100° C. for 45 min.

The elemental analysis of the catalyst shaped body showed the following proportions:
Pd: 1.3% by weight
Au: 0.43% by weight

Determination of the noble metal distribution was carried out as in example 1. The maximum of the Pd concentration and the maximum of the Au concentration were 30 micrometers below the geometric macroscopic surface of the shell catalyst. The shell thickness for Pd was 129 micrometers and for Au was 75 micrometers. FIG. 5 shows both the curves of the Pd and Au concentrations based on their fitted function, wherein for better comparability the values of the Au concentration have been multiplied by a factor of 5.

### Comparative Example 1: Catalyst 3

100 g of the bentonite-containing support material KA-160 (available from Clariant) were weighed in and impregnated with a mixture of 39.3 g of 2 molar KOAc solution and 18.1 g deionized water according to the pore filling method (incipient wetness). After static drying in the fluidized bed dryer at 90° C. for 35 min the mixture was cooled to room temperature and transferred to an Innojet IAC 025 coater. By means of a process gas the KOAc-impregnated support was set into a fluidized bed motion and heated to 70° C. and the temperature maintained for 2 minutes before spray-application of a cesium laurate solution was commenced. To this end 7.0 g of an aqueous cesium aurate solution (4.7% by weight Au) were diluted with deionized water to afford 50 g of coating solution and said solution was applied to the catalyst support in a first coating step at a spray rate of 4 g/min in the coater, the temperature of the process air falling by 15° C. This was increased to 71° C. by active temperature control and maintained at this temperature. Subsequently in a second coating step a mixture of 2.4 g of an aqueous cesium aurate solution (4.7% by weight Au) and 38.9 g of a tetraammine palladium dihydroxide solution (3.48 by weight Pd) was diluted with deionized water to afford 80 g of coating solution and applied to the catalyst supports at a spray rate of 4 g/min and a process air temperature of 70° C. The catalyst supports continue to be kept in a fluidized bed. After a further static drying in the fluidized bed dryer (90° C./35 min) the catalyst was subjected to static reduction in a tubular furnace with forming gas (2% H2 in N2) at 100° C. for 45 min.

The elemental analysis of the catalyst showed the following proportions:
Pd: 1.3% by weight
Au: 0.43% by weight

Determination of the noble metal distribution was carried out as in example 1. The maximum of the Pd concentration was at 43 micrometers and the maximum of the Au concentration was 31 micrometers below the surface of the shell catalyst. The shell thickness for Pd was 183 micrometers and for Au was 148 micrometers. FIG. 6 shows both the curves of the Pd and Au concentrations based on their fitted functions, wherein for better comparability the values of the Au concentration have been multiplied by a factor of 8.

### Example 3: Reactor Test

Test results of catalysts 1, 2 and 3 having regard to their activity, selectivity and productivity in the synthesis of vinyl acetate monomer:

For the catalytic tests in each case 2.9 g of catalyst were filled into a reactor having a volume of 5.7 ml and then heated to 138° C. under an inert gas. At this temperature the inert gas stream was replaced by a stream of acetic acid, ethylene and oxygen that was passed through the reactor. At regular intervals, downstream of the reactor, samples of the output streams were withdrawn and analyzed by gas chromatography. After a reaction time of 24 hours at 138° C. the reactor temperature was increased to 140° C. and maintained for a further 12 hours. This was followed by further temperature increases to 142° C., 144° C., 146° C. and finally back to 140° C. The respective reaction times were 12 hours in each case and the pressure 5-6 barg. The concentrations of the employed components were: 45% ethylene, 6% O₂, 0.9% CO₂, 9% methane, 15.5% acetic acid, residual N₂.

Table 1 and FIG. 7 show the selectivity/the activity of catalysts 1, 2 and 3 as a function of O₂ conversion. It is clearly apparent therefrom that the catalysts 1 and 2 produced according to the invention have a much higher activity, selectivity (at the same activity level) and productivity than the comparative catalyst catalyst 3. This is additionally elucidated in FIG. 7 which shows a plot of selectivity for VAM as a function of space-time yield.

TABLE 1
Catalytic test data of catalysts 1 to 3

| | Temperature | | | | | | |
|---|---|---|---|---|---|---|---|
| Example | [° C.] | 138 | 140 | 142 | 144 | 146 | 140 |
| | Pressure | 6 | 6 | 5 | 5 | 5 | 5 |
| | [barg] | | | | | | |
| Catalyst 1 | Yield | 823 | 874 | 825 | 861 | 894 | 766 |
| | [g/(L · h)] | | | | | | |
| | Selectivity | 96.7 | 96.7 | 96.7 | 96.5 | 96.4 | 97.2 |
| | [%] | | | | | | |
| | O₂ | 73.4 | 76.9 | 72.4 | 75.8 | 79.7 | 65.1 |
| | conversion | | | | | | |
| | [%] | | | | | | |
| Catalyst 2 | Yield | 808 | 854 | 808 | 843 | 882 | 738 |
| | [g/(L · h)] | | | | | | |
| | Selectivity | 96.8 | 96.8 | 96.8 | 96.7 | 96.5 | 97.3 |
| | [%] | | | | | | |
| | O₂ | 71.2 | 74.6 | 70.4 | 73.9 | 77.2 | 62.1 |
| | conversion | | | | | | |
| | [%] | | | | | | |
| Catalyst 3 | Yield | 734 | 783 | 738 | 772 | 809 | 685 |
| | [g/(L · h)] | | | | | | |
| | Selectivity | 96.9 | 96.9 | 96.9 | 96.7 | 96.6 | 97.3 |
| | [%] | | | | | | |
| | O₂ | 64.8 | 68.7 | 64.4 | 68.3 | 71.1 | 57.8 |
| | conversion | | | | | | |
| | [%] | | | | | | |

1. A Pd- and Au-containing shell catalyst wherein it has a concentration profile of the palladium and of the gold within the shell catalyst where the maximum of the palladium concentration and the maximum of the gold concentration are disposed in a range from 0 to 40 micrometers from the geometric macroscopic surface of the catalyst shaped body and the distance between the maximum of the palladium concentration and the maximum of the gold concentration is in the range from 0 to 10 micrometers.

2. The shell catalyst as claimed in claim 1, wherein the maximum of the Pd concentration and the maximum of the Au concentration are in the range from 5 to 40 micrometers, preferably 5 to 35 micrometers, particularly preferably 5 to 30 micrometers, from the surface of the shell catalyst.

3. The shell catalyst as claimed in claim 1, wherein the distance between the maximum of the palladium concentration and the gold concentration is in the range from 0 to 8 micrometers, preferably in the range from 0 to 5 micrometers.

4. The shell catalyst as claimed in claim 1, wherein the maximum of the Pd concentration is closer to the geometric surface than the maximum of the Au concentration.

5. The shell catalyst as claimed in claim 1, wherein the shell comprising Pd and Au has a thickness of 30 to 200 micrometers, preferably 40 to 180 micrometers, particularly preferably 50 to 160 micrometers, most preferably 60 to 140 micrometers.

6. The shell catalyst as claimed in claim 1, further comprising an alkali metal acetate, preferably potassium acetate.

7. The shell catalyst as claimed in claim 1, wherein the proportion of Pd is in the range from 0.2% to 2.0% by weight, preferably in the range from 0.4% to 1.75% by weight and particularly preferably in the range from 0.7% to 1.5% by weight and the proportion of Au is in the range from 0.1% to 1.2% by weight, preferably in the range from 0.2% to 1.0% by weight and most preferably in the range from 0.3% to 0.8% by weight, in each case based on the total weight of the catalyst shaped body after reduction and loss on drying.

8. A process for producing a Pd- and Au-containing shell catalyst as claimed in claim 1, comprising the steps of:
(a) subjecting a bed of a catalyst support to a recirculating motion, wherein the bed is heated to a temperature of 60° C. to 120° C. before or after subjection to the recirculating motion,
(b) sequential or simultaneous application of a dissolved Pd-containing precursor compound and a dissolved Au-containing precursor compound by spray application onto the bed of the catalyst support subjected to the recirculating motion, wherein in the case of simultaneous application the temperature is 5° C. to 30° C., preferably 7° C. to 25° C., most preferably 10° C. to 20° C., below the temperature established in step (a) and wherein in the case of sequential application the temperature of the first application is 5° C. to 30° C., preferably 7° C. to 25° C., most preferably 10° C. to 20° C., below the temperature established in step (a).

9. The process as claimed in claim 8, wherein step (b) is followed by a step (c) comprising reduction of the metal components of the precursor compounds to the elemental metals by subjecting the catalyst support body obtained in step (b) to a heat treatment in a non-oxidizing atmosphere.

10. The process as claimed in claim 8, wherein the temperature of the application or in the case of sequential application the temperature of the first application in step (b) is in the range from 55° C. to 115° C., preferably in the range from 55° C. to 110° C., more preferably in the range from 60° C. to 100° C., particularly preferably in the range from 65° C. to 90° C.

11. The process as claimed in claim 8, wherein in step (b) in a first step a dissolved Au-containing precursor compound is applied and in a second step a dissolved Pd-containing precursor compound is applied.

12. A process for producing a Pd- and Au-containing shell catalyst as claimed in claim 1, comprising the steps of:
(a) subjecting a bed of a catalyst support to recirculating motion and contacting the bed with atomized water by spray application at a temperature in the range from 55° C. to 110° C.;
(b) sequential or simultaneous application of a dissolved Pd-containing precursor compound and a dissolved Au-containing precursor compound by spray application onto the bed of the catalyst support subjected to the recirculating motion, wherein in the case of simultaneous application the temperature is the same as in step (a) and wherein in the case of sequential application the temperature of the first application is the same as in step (a).

13. The process as claimed in claim 12, wherein step (b) is followed by a step (c) comprising reduction of the metal components of the precursor compounds to the elemental metals by subjecting the catalyst support body obtained in step (b) to a heat treatment in a non-oxidizing atmosphere.

14. The process as claimed in claim 12, wherein in step (b) in a first step a dissolved Au-containing precursor compound and in a second step a dissolved Pd-containing precursor compound is applied.

15. A process for producing alkenyl carboxylic esters, in particular VAM or allyl acetate monomer, with the shell catalyst as claimed in claim 1.
